(19) United States
(12) Patent Application Publication
(10) Pub. No.: US 2019/0201249 A1
(43) Pub. Date: Jul. 4, 2019

(54) FOAM TAMPON

(71) Applicant: George Moore, Atlanta, GA (US)

(72) Inventor: George Moore, Atlanta, GA (US)

(21) Appl. No.: 15/862,524

(22) Filed: Jan. 4, 2018

(51) Int. Cl. A61F 13/20

(52) U.S. Cl. CPC A61F 13/2057; A61F 13/26; A61F 13/2022

(57) ABSTRACT

Implementations of a foam tampon are provided. In some implementations, the foam tampon comprises a foam body. In some implementations, the foam body is a single layer made from foam and configured to have an octagon shape. In some implementations, the foam tampon further comprises a string attached to and extending from the single layer foam body. In some implementations, the foam tampon further comprises a tubular container wherein the foam body is folded lengthwise and housed inside the tubular container. In some implementations, the foam tampon includes no other layers of materials to restrict the natural expansion from a compacted condition of the single layer foam body when the single layer foam body is no longer compressed.

### TECHNICAL FIELD

This disclosure relates to implementations of a foam tampon.

### BACKGROUND

U.S. Pat. Nos. 3,834,389 and 3,794,029 disclose foam tampon of a particular shape to have a particular orientation in the vagina when inserted with the disclosed applicators.

### BRIEF DESCRIPTION OF THE DRAWINGS

FIG. 1 illustrates an implementation of an example foam body of a foam tampon according to the present disclosure.

FIG. 2 illustrates an example front view of the foam body of FIG. 1 with a string attached thereto.

FIGS. 3A-3C illustrate an example tubular container for storing the foam body of FIG. 1.

### DETAILED DESCRIPTION

Implementations of foam tampons are provided. In some implementations, the foam tampon comprising a single layer body made from foam. In some implementations, the foam tampon consist of a single layer body made from foam. In some implementations, the foam tampon is configured such that the single layer foam body is unrestricted from expanding naturally from a compacted condition when the single layer foam body is no longer compressed. In some implementations, the foam tampon includes no other layers of materials to restrict the natural expansion from a compacted condition of the single layer foam body when the single layer foam body is no longer compressed.

In some implementations, the single layer body is polyurethane foam. In some implementations, the single layer foam body is absorbent. In some implementations, the single layer foam body is open cell foam. In some implementations, the single layer foam body is configured to expand naturally from a compacted condition toward its original shape when the single layer foam body is no longer compressed. In some implementations, the single layer foam body is hypoallergenic. In some implementations, the single layer foam body is non-toxic. In some implementations, the single layer foam body is latex free. In some implementations, the single layer foam body is configured for comfortable housing in the vagina. In some implementations, the single layer foam body is flexible. In some implementations, the single layer foam body is resilient. In some implementations, the single layer foam body is mensesphilic. In some implementations, the single layer foam body is hydrophilic. In some implementations, the single layer foam body has a modulus of compression such that the vaginal walls may compress the single layer foam body.

In some implementations, the single layer foam body may be configured to have an octagon-shaped first surface, an octagon-shaped second surface, opposite the first surface, and eight rectangular sides extending from the first surface to the second surface. In some implementations, the single layer foam body is elongated. In some implementations, the length of the single layer foam body is greater than its width.

In some implementations, the foam tampon further comprises a string having a first end and a second end attached to and extending from the single layer foam body. In some implementations, the foam tampon consists of a single layer foam body and a string attached to and extending from the single layer foam body. In some implementations, the string is threaded through the single layer foam body to attach the string to the single layer foam body. In some implementations, the string is threaded lengthwise through the single layer foam body from one side of the single layer foam body toward an opposite side of the single layer foam body.

In some implementations, a first end of the string is inserted into the single layer foam body at a first rectangular side of the single layer foam body and threaded through the single layer foam body lengthwise towards a second rectangular side, opposite the first rectangular side, of the single layer foam body and then back towards the first rectangular side of the single layer foam body where the first end of the string exits the single layer foam body at the first rectangular side. In some implementations, the string may be attached to the single layer foam body in any suitable manner. In some implementations, the string may be made from cotton.

In some implementations, the foam body is stored in a tubular container for insertion into a vagina. In some implementations, the foam body is folded lengthwise while housed in the tubular container.

In some implementations, the tampon is configured so that the single layer foam body expands naturally (i.e., dry expands) toward its original shape until it reaches its maximum dry expansion or is restricted by the vagina when the tampon is ejected from the tubular container when the tubular container is inserted inside a vagina.

In some implementations, the tampon is sized so that the single layer foam body expands naturally toward its original shape until it is restricted by the vagina before it reaches its maximum dry expansion when the tampon is ejected from the tubular container when the tubular container is inserted inside a vagina.

In this way, the foam tampons of the present disclosure may provide better comfort and absorption than existing foam tampons.

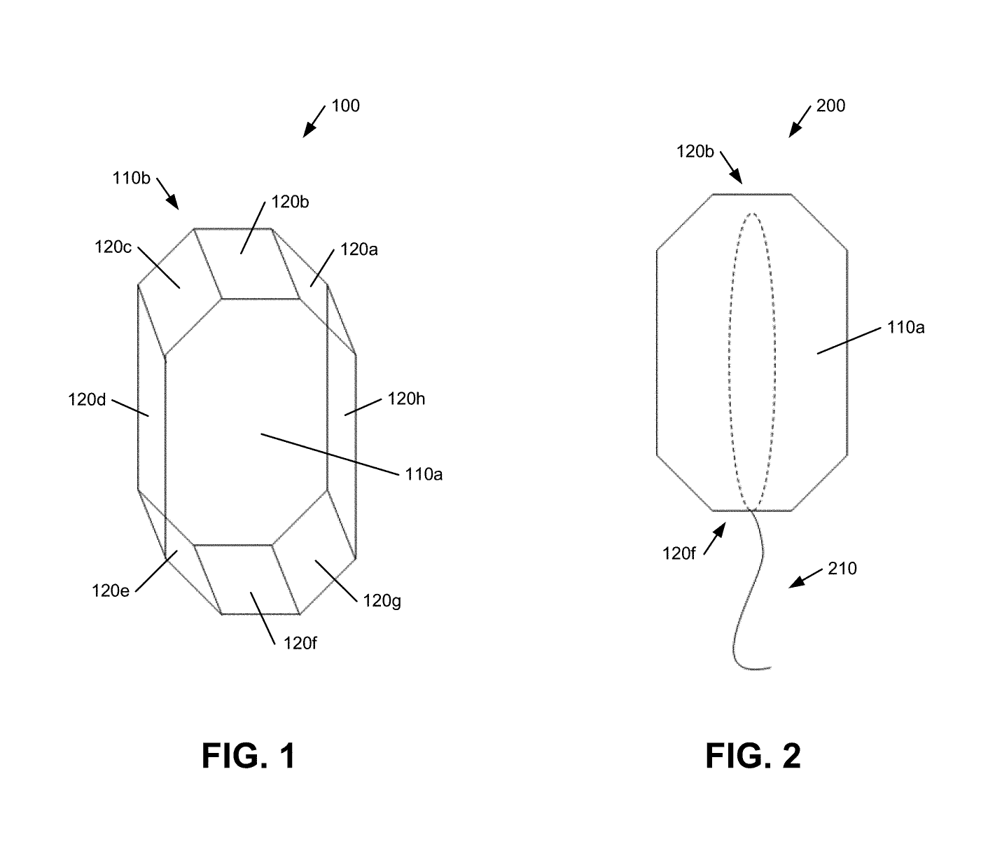

FIG. 1 illustrates an implementation of an example foam body 100 of a foam tampon according to the present disclosure.

As discussed above, in some implementations, the single layer foam body 100 may be configured to have an octagon-shaped first surface 110a, an octagon-shaped second surface 110b, opposite the first surface, and eight rectangular sides 120a-h extending from the first surface 110a to the second surface 110b.

In some implementations, the single layer foam body is elongated. In some implementations, the length of the single layer foam body is greater than its width.

As discussed above, in some implementations, the foam body (e.g., body 100) is a single layer body. In some implementations, the single layer body is made from foam.

As discussed above, in some implementations, the single layer foam body is unrestricted from expanding naturally from a compacted condition when the single layer foam body is no longer compressed.

In some implementations, the single layer body is polyurethane foam. In some implementations, the single layer foam body is absorbent. In some implementations, the single layer foam body is open cell foam.

In some implementations, the single layer foam body is configured to expand naturally from a compacted condition toward its original shape when the single layer foam body is no longer compressed.

In some implementations, the single layer foam body is hypoallergenic.

In some implementations, the single layer foam body is configured for comfortable housing in the vagina.

In some implementations, the single layer foam body is a flexible.

In some implementations, the single layer foam body is resilient.

In some implementations, the single layer foam body is mensesphilic.

In some implementations, the single layer foam body is hydrophilic.

In some implementations, the single layer foam body has a modulus of compression such that the vaginal walls may compress the single layer foam body.

FIG. 2 illustrates an example front view of the foam body of FIG. 1 with a string 210 attached thereto.

As discussed above, in some implementations, the string 210 is threaded through the single layer foam body (e.g., body 100) to attach the string to the single layer foam body. In some implementations, the string 210 is threaded lengthwise through the single layer foam body from one side of the single layer foam body toward an opposite side of the single layer foam body.

For example, in some implementations, a first end of the string 210 is inserted into the single layer foam body at a first rectangular side (e.g., side 120f of the single layer foam body and threaded through the single layer foam body lengthwise towards a second rectangular side (e.g., side 120b), opposite the first rectangular side, of the single layer foam body and then back towards the first rectangular side of the single layer foam body where the first end of the string exits the single layer foam body at the first rectangular side.

In some implementations, the string 210 may be attached to the single layer foam body in any suitable manner. Those of ordinary skill in the art know other ways to securely attach the string to the foam body and this disclosure is not limited to any particular existing or future developed method of securing the string to the foam. The string may be attached to the foam body using any existing or future developed method.

In some implementations, the string may be made from cotton. In some implementations, the string may be any suitable material.

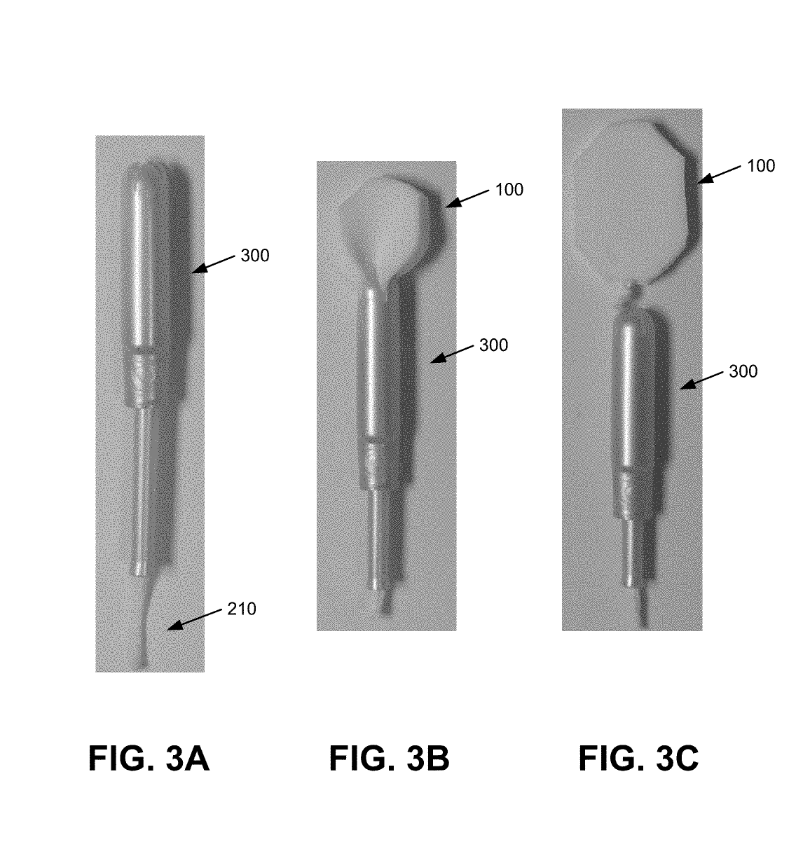

FIGS. 3A-3C illustrate an example tubular container 300 for storing the foam body 100 of FIG. 1.

FIG. 3A illustrates the form body 100 compressed inside the tubular container 300.

FIG. 3B illustrates the form body 100 partially ejected from the tubular container 300. As shown in FIG. 3B and discussed in above, in some implementations, the foam body 100 is folded lengthwise while housed in the tubular container 300.

FIG. 3C illustrates the form body 100 fully ejected from the tubular container 300. In some implementations, the form body 100 may not expand back to its original shape. As discussed above, in some implementations, the tampon is configured so that the single layer foam body expands naturally (i.e., dry expands) toward its original shape until it reaches its maximum dry expansion or is restricted by the vagina when the tampon is ejected from the tubular container when the tubular container is inserted inside a vagina.

As discussed above, in some implementations, the foam tampon includes no other layers of materials to restrict the natural expansion from a compacted condition of the single layer foam body 100 when the single layer foam body is no longer compressed.

In some implementations, the tampon is sized so that the single layer foam body expands naturally toward its original shape until it is restricted by the vagina before it reaches its maximum dry expansion when the tampon is ejected from the tubular container when the tubular container is inserted inside a vagina.

As shown in FIG. 3B, in some implementations, the foam body is folded or rolled up along or around a line that extends in the same direction in which the string runs through the foam body. The folded or rolled up foam then is inserted in the tubular container 300 such that when the foam body 100 is inserted into the vagina using the tubular container 300, the foam body 100 emerges in the vagina in the orientation and general shape shown in FIG. 3C wherein the string 201 will hang outside the body. As discussed above, in some implementations, the foam body 100 may be compressed in the vagina due to the vaginal walls.

The thickness of the foam can vary and this disclosure is not limited to any particular thickness.

Reference throughout this specification to “an embodiment” or “an implementation” or words of similar import means that a particular described feature, structure, or characteristic is included in at least one embodiment of the present invention. Thus, the phrase “in an embodiment” or “an implementation” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

1. A foam tampon comprising:
a foam body wherein the foam body is a single layer made from foam wherein the single layer foam body is configured to have an octagon-shaped first surface, an octagon-shaped second surface, opposite the first surface, and eight rectangular sides extending from the first surface to the second surface; wherein the single layer foam body is elongated; and
string attached to and extending from the single layer foam body;
wherein the foam tampon includes no other layers of materials to restrict the natural expansion from a compacted condition of the single layer foam body when the single layer foam body is no longer compressed.

2. The foam tampon of claim 1 wherein the foam body is polyurethane foam.

3. The foam tampon of claim 1 wherein the foam body is configured for housing in a vagina.

4. The foam tampon of claim 1 wherein the foam body has a modulus of compression such that vaginal walls compress the foam body.

5. The foam tampon of claim 1 wherein the string is threaded lengthwise through the foam body at a first rectangular side of the foam body towards a second rectangular side, opposite the first rectangular side, of the foam body and then back towards the first rectangular side of the foam body where the first rectangular side is at a bottom of the foam body and the second rectangular side is at a top of the foam body.

6. The foam tampon of claim 1 further comprising a tubular container wherein the foam body is folded lengthwise and housed inside the tubular container.

7. A foam tampon comprising:
a foam body wherein the foam body is a single layer made from foam wherein the single layer foam body is configured to have an octagon-shaped first surface, an octagon-shaped second surface, opposite the first surface, and eight rectangular sides extending from the first surface to the second surface; wherein the single layer foam body is elongated;
string attached to and extending from the single layer foam body wherein the string is threaded lengthwise through the foam body at a first rectangular side of the foam body towards a second rectangular side, opposite the first rectangular side, of the foam body and then back towards the first rectangular side of the foam body where the first rectangular side is at a bottom of the foam body and the second rectangular side is at a top of the foam body; and
a tubular container wherein the foam body is folded lengthwise and housed inside the tubular container,
wherein the foam tampon includes no other layers of materials to restrict the natural expansion from a compacted condition of the single layer foam body when the single layer foam body is no longer compressed.
